(19) United States
(12) Patent Application Publication
(10) Pub. No.: US 2016/0000345 A1
(43) Pub. Date: Jan. 7, 2016

(54) MONITORING DEVICE AND MONITORING DEVICE KIT

(71) Applicant: TERUMO KABUSHIKI KAISHA, Tokyo (JP)

(72) Inventors: Risato KOBAYASHI, Yokohama-city (JP); Wataru KARINO, Atsugi-city (JP); Ichirou HIRAHARA, Tokyo (JP); Junichi KOBAYASHI, Cupertino, CA (US)

(73) Assignee: TERUMO KABUSHIKI KAISHA, Tokyo (JP)

(21) Appl. No.: 14/857,867

(22) Filed: Sep. 18, 2015

Related U.S. Application Data
(63) Continuation of application No. PCT/JP2014/001253, filed on Mar. 6, 2014.

(30) Foreign Application Priority Data
Apr. 1, 2013 (JP) 2013-076425

(51) Int. Cl. A61B 5/04; A61B 5/00

(52) U.S. Cl. CPC A61B 5/04001; A61B 5/6862; A61B 5/4848

(57) ABSTRACT

A monitoring device includes an expansion body that is delivered into a vessel, and that expands at a predetermined position, and an electrode that is attached to the expansion body, and that detects neural activities of a nerve located outside the vessel by coming into contact with an inner wall of the vessel when the expansion body expands.

### CROSS-REFERENCES TO RELATED APPLICATIONS

This application is a continuation of International Application No. PCT/JP2014/001253 filed Mar. 6, 2014, and claims priority to JP2013-076425 filed Apr. 1, 2013, the entire content of each of which is incorporated herein by reference.

### TECHNICAL FIELD

The present invention relates to a monitoring device and a monitoring device kit.

### BACKGROUND DISCUSSION

In the medical field, an elongated hollow tube called a catheter is used in performing various types of medical treatments and examinations. Examples of such medical treatment methods include a method of directly administering a drug to an affected site through a catheter, a method of pressing, widening, and opening a stenosis in a body lumen by using a catheter in which a dilatable balloon is attached to a distal end thereof, a method of scraping and opening the affected site using the catheter in which a cutter is attached to a distal end portion thereof, and the like.

When treatment is performed using a catheter at a lesion site in, for example, a blood vessel, the catheter is percutaneously inserted into the lesion site from a punctured portion formed in the arm or the leg. Then, a treatment device is inserted into the lesion site through the catheter so as to perform the treatment. One such treatment inactivates a sympathetic nerve of the renal artery for a resistant hypertensive patient. Hereinafter, inactivating a nerve is referred to as “denervation”.

### SUMMARY

With regard to treatment for performing a denervation as described above, a determination method of determining whether or not the denervation has been reliably performed during the treatment or immediately after the treatment has not yet been established. Consequently, it can be difficult to determine whether or not additional treatment is needed for a patient who shows no treatment effect even after the treatment is performed. In addition, there are other treatments which would benefit from monitoring neural activities during the treatment or immediately after the treatment.

In view of the foregoing, an object of the present disclosure is to provide a monitoring device and a monitoring device kit which can monitor neural activities during treatment or immediately after treatment.

In order to achieve the above-described object and other objects, a monitoring device according to a first aspect of the present disclosure includes an expansion body that is delivered into a vessel, and that expands at a predetermined position, and an electrode that is attached to the expansion body, and that detects neural activities of a nerve located outside the vessel by coming into contact with an inner wall of the vessel when the expansion body expands.

According to one embodiment of the present disclosure, the electrode is disposed at multiple locations, and the multiple electrodes come into contact with the inner wall of the vessel at mutually different positions in a circumferential direction of the vessel.

According to one embodiment of the present disclosure, the device includes an identification member that can identify each of the multiple electrodes.

According to one embodiment of the present disclosure, the expansion body partitions a hollow portion through which a body fluid can pass.

According to one embodiment of the present disclosure, the expansion body is a self-expandable stent which has a cylindrical shape, and the electrode is attached onto an outer peripheral surface of the stent in an expanded state.

According to one embodiment of the present disclosure, a hook member is disposed in one end portion of the stent.

According to one embodiment of the present disclosure, the expansion body is a balloon which is dilated by a liquid being supplied to an annular cavity portion partitioned there inside, and the electrode is attached onto an outer peripheral surface of the balloon in a dilated state.

According to one embodiment of the present disclosure, the electrode is disposed at multiple locations along an extending direction of the vessel.

According to one embodiment of the present disclosure, the electrode is a rectangular-shaped electrode which extends along the extending direction of the vessel.

According to one embodiment of the present disclosure, the expansion body is a spiral shape memory alloy, and the electrode is attached to a surface of the shape memory alloy.

According to one embodiment of the present disclosure, a hook member is disposed in one end portion of the shape memory alloy.

According to one embodiment of the present disclosure, the electrode includes an elastically deformable projection portion which is attached to the expansion body and a detecting element which is attached to a distal end of the projection portion, and in a state where the expansion body expands, the projection portion is elastically deformed, and the detecting element is pressed against the inner wall of the vessel.

According to one embodiment of the present disclosure, the vessel is a blood vessel, and a protective filter is attached to a downstream side end portion in a blood flowing direction of the expansion body in a state where the expansion body indwells the blood vessel.

A monitoring device kit according to a second aspect of the present disclosure includes the monitoring device and a delivery member that accommodates the expansion body in a state where the maximum length of the expansion body in a radial direction of the vessel is shorter than the maximum length in a state where the expansion body expands at the predetermined position, and that can deliver the expansion body to the predetermined position.

According to the present disclosure, it is possible to monitor neural activities during treatment or even immediately after treatment.

### BRIEF DESCRIPTION OF THE DRAWINGS

FIG. 1 is a perspective view illustrating a state where a monitoring device 1 according to a first embodiment indwells a vessel VE.

FIG. 2 is a view for describing treatment for performing denervation on a sympathetic nerve of the renal artery.

FIG. 3 is a perspective view illustrating a state where a monitoring device 11 according to a second embodiment indwells the vessel VE.

FIG. 4 is a perspective view illustrating a state where a monitoring device 21 according to a third embodiment indwells the vessel VE.

FIG. 5 is a perspective view illustrating a state where a monitoring device 31 according to a fourth embodiment indwells the vessel VE.

FIG. 6 is a perspective view illustrating a state where a monitoring device 41 according to a fifth embodiment indwells the vessel VE.

FIG. 7 is a perspective view illustrating a state where a monitoring device 51 according to a sixth embodiment indwells the vessel VE.

FIG. 8 is a perspective view illustrating a state where a monitoring device 61 according to a seventh embodiment indwells the vessel VE.

FIG. 9 is a sectional view illustrating a monitoring device kit 101 according to an eighth embodiment.

FIG. 10 is a perspective view illustrating the monitoring device 11 being delivered into the vessel VE.

### DETAILED DESCRIPTION

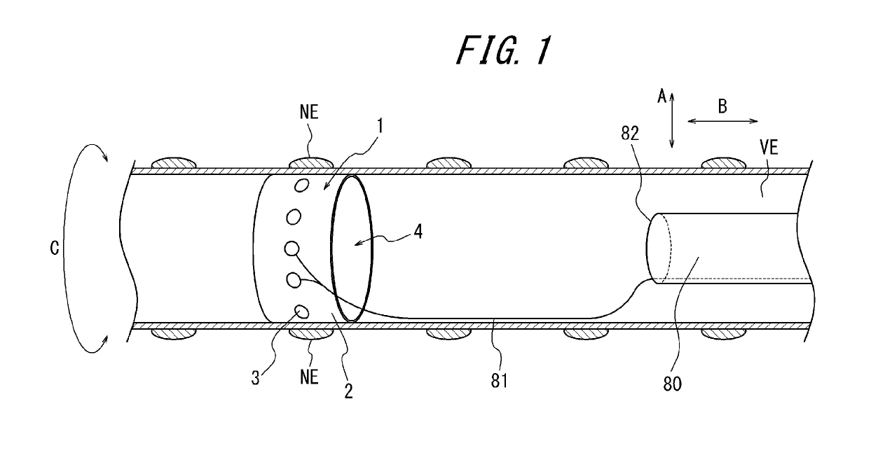

Hereinafter, embodiments of a monitoring device and a monitoring device kit will be described with reference to FIGS. 1 to 10. The same reference numerals are given to common members in each drawing.

First, Embodiment 1 which is one embodiment of the monitoring device will be described. FIG. 1 is a view illustrating a monitoring device 1 according to the present embodiment.

As illustrated in FIG. 1, the monitoring device 1 according to the present embodiment includes an expansion body 2 which expands at a predetermined position inside a vessel VE, and an electrode 3 which is attached to the expansion body 2, and which detects neural activities of a nerve NE located outside the vessel VE by coming into contact with an inner wall of the vessel VE when the expansion body 2 expands. The vessel VE is a duct which is present inside a body for passage of body fluid therethrough. For example, the vessel VE can include a blood vessel, a lymph duct, and the like.

Hereinafter, details of each member element of the monitoring device 1 according to the present embodiment will be described.

In a contracted state or in a folded state, that is, in a size-reduced state where the maximum length of the expansion body 2 in a radial direction A of the vessel VE is shorter than the maximum length in an expanded state at a predetermined position, the expansion body 2 is delivered to the predetermined position inside the vessel VE from the outside of a body through a guiding catheter 80, and expands so as to indwell at the predetermined position. FIG. 1 illustrates a state where the expansion body 2 expands and indwells at the predetermined position.

The expansion body 2 according to the present embodiment partitions a substantially cylindrical hollow portion 4 in a manner in which a body fluid inside the vessel VE can flow to a downstream side (left side in FIG. 1) in an extending direction B of the vessel VE after passing through the expansion body 2, even in a state where the expansion body 2 expands and indwells the vessel VE. According to this configuration, the body fluid can pass through the hollow portion 4. Accordingly, it is possible to maintain a flow of the body fluid even after the monitoring device 1 indwells, and thus minimize a load applied to a human body during use of the device. If the expansion body 2 is used for treatment to be completed in a short period of time, the hollow portion 4 may not need to be partitioned. That is, the expansion body 2 could be employed in a shape in which the flow of the body fluid is blocked (for example, a cylindrical solid shape). However, the expansion body may be configured such that, regardless of a time for treatment, the hollow portion is partitioned during use so that body fluid can pass therehrough.

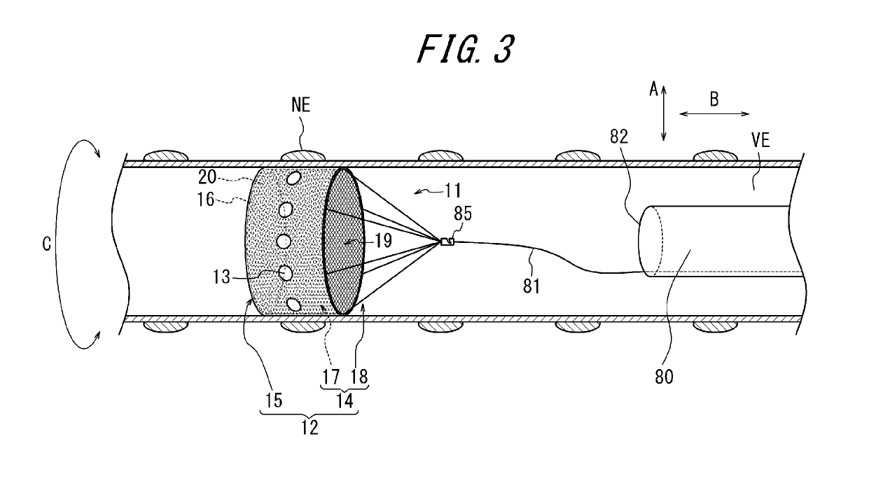

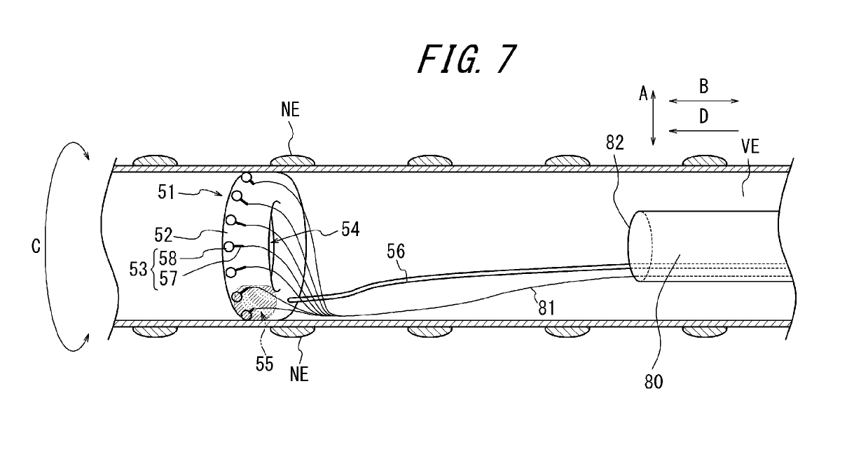

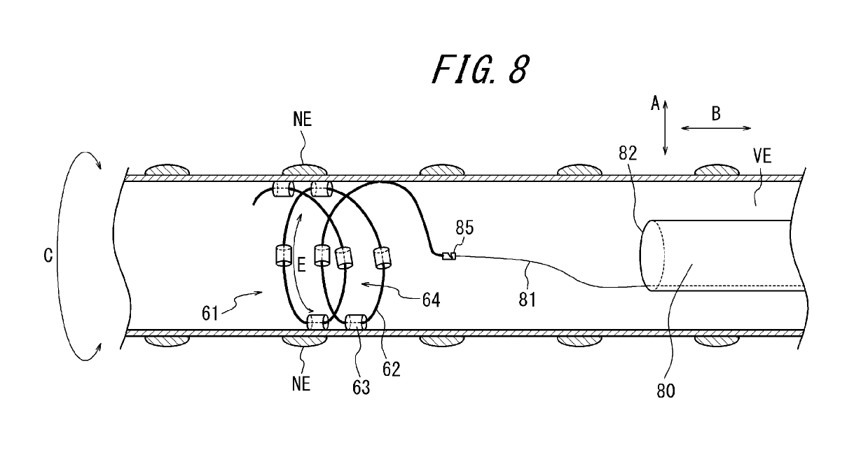

The expansion body 2 according to the present embodiment may have a cylindrical hollow shape in an expanded state. For example, the expansion body 2 can be configured to include a self-expandable stent (refer to FIGS. 3 to 6) and a balloon (refer to FIG. 7) which will be described later as another embodiment. In addition, the expansion body can be configured to include a spiral shape memory alloy (refer to FIG. 8) or the like which is formed in a substantially cylindrical hollow shape as a whole.

The electrode 3 is attached to the expansion body 2, and is delivered to a predetermined position inside the vessel VE through the guiding catheter 80, together with the expansion body 2. When the expansion body 2 expands at the predetermined position, the electrode 3 comes into contact with an inner wall of the vessel VE.

The electrode 3 is formed of one or more metals such as platinum, iridium, tungsten, and the like, for example, and is attached to a surface of the expansion body 2 by means of bonding or vapor deposition, for example. In addition, the electrode 3 can be configured to include a flexible conductive polymer.

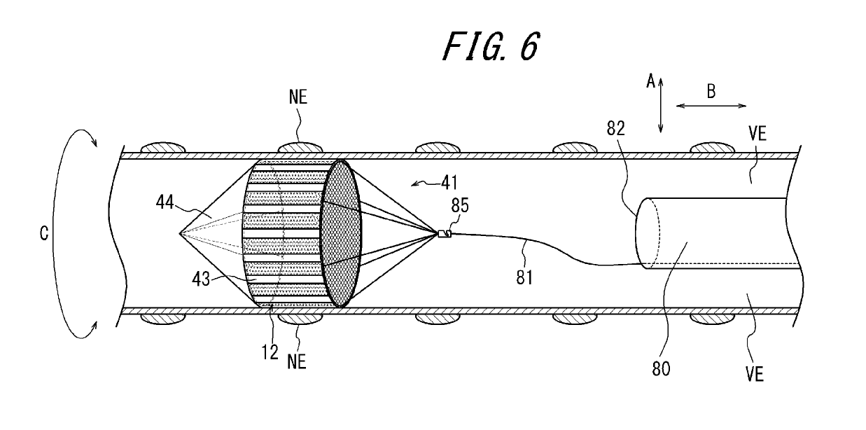

The electrode 3 according to the present embodiment has a substantially circular flat shape. However, for example, depending on the shape of the expansion body 2, the electrode 3 can have various shapes such as a substantially rectangular flat shape (refer to FIG. 6), a small and substantially spherical shape (refer to FIG. 7), a columnar shape (refer to FIG. 8), and the like, as will be described later in another embodiment.

In a state where the expansion body 2 expands, the electrode 3 according to the present embodiment is disposed at multiple locations in a circumferential direction of the expansion body 2. The multiple electrodes 3 are in contact with the inner wall of the vessel VE at mutually different positions in a circumferential direction C (here, the same direction as the circumferential direction of the expansion body 2) of the vessel VE. In this manner, the nerve NE whose neural activities are detectable can be found with a higher probability.

As the distance between the electrode 3 and the measurement target nerve NE becomes closer, detection sensitivity is further improved, thereby enabling accurate monitoring. Accordingly, it is preferable that the distance between the electrode 3 and the measurement target nerve NE is 10 mm or shorter in a state where the electrode 3 indwells after coming into contact with the inner wall of the vessel VE.

In addition, each electrode 3 has a wire 81 connected thereto, and each wire 81 is connected to a measuring device located outside the body through the guiding catheter 80. This enables the measuring device located outside the body to observe the neural activities of the nerve NE. In addition, the wires 81 connected to each electrode 3 are configured to be bundled into one in a region where the monitoring device 1 is present in the extending direction B of the vessel VE as illustrated in FIG. 1, or in a region between the monitoring device 1 and a distal end 82 of the guiding catheter 80. In this manner, the wire 81 is less likely to interfere with treatment. For clarity purposes, FIG. 1 illustrates only some of the electrodes 3 connected to a wire 81, but all of the electrodes 3 will be connected to a wire 81 in practice. In addition, the neural activities of the nerve NE which are detected by the electrode 3 can also be wirelessly transmitted to the measuring device located outside the body by a transmitter (not illustrated) attached to the expansion body 2, instead of by wire. In this manner, it is possible to efficiently supply a treatment device to a treatment target region during treatment.

Here, a “predetermined position”, which is a position where the expansion body 2 expands, is at least necessarily any position inside the vessel VE where the electrode 3 can detect the neural activities of the measurement target nerve NE located outside the vessel VE. However, the specific position of the expansion body 2, selected from the possible range of predetermined positions, will depend on the type of treatment. Such a specific position will be described below by using an example of treatment for performing denervation on a sympathetic nerve in the renal artery.

Hitherto, the configuration of each member in the monitoring device 1 has been mainly described. Hereinafter, as one method of use of the monitoring device 1 according to the present embodiment, a method of use in treatment for performing denervation on a sympathetic nerve in the renal artery will be described.

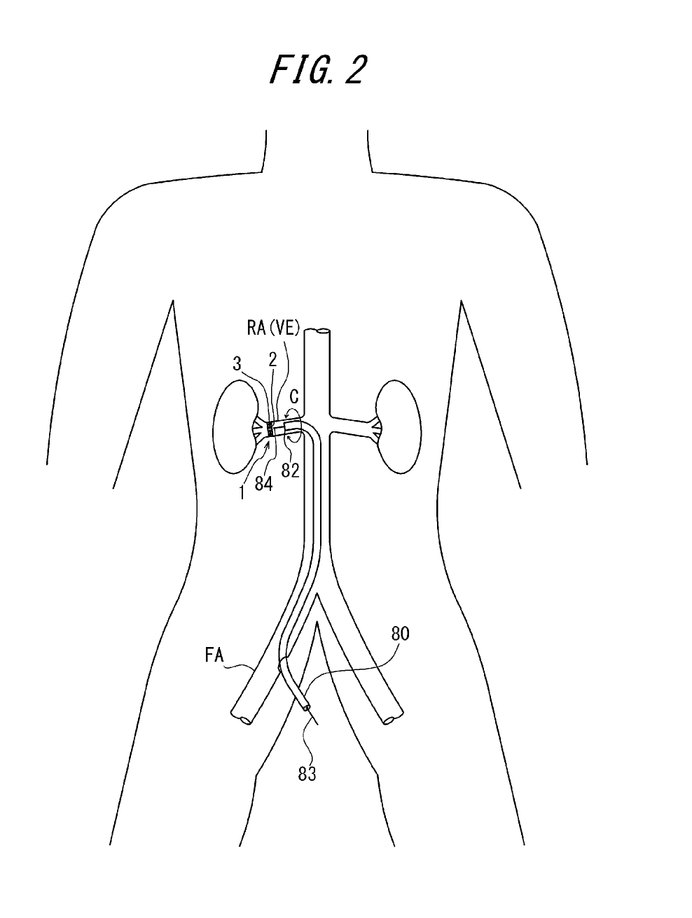

FIG. 2 is a view for describing the treatment for performing denervation on the sympathetic nerve of the renal artery. An operator inserts the guiding catheter 80 into a femoral artery FA of a patient in advance, and causes the distal end 82 of the guiding catheter 80 to reach a renal artery RA. A guidewire (not illustrated) is used in order to cause the guiding catheter 80 to reach the renal artery RA.

The guiding catheter 80 has a tubular shape, and the treatment device or the monitoring device 1 can be inserted into the guiding catheter 80.

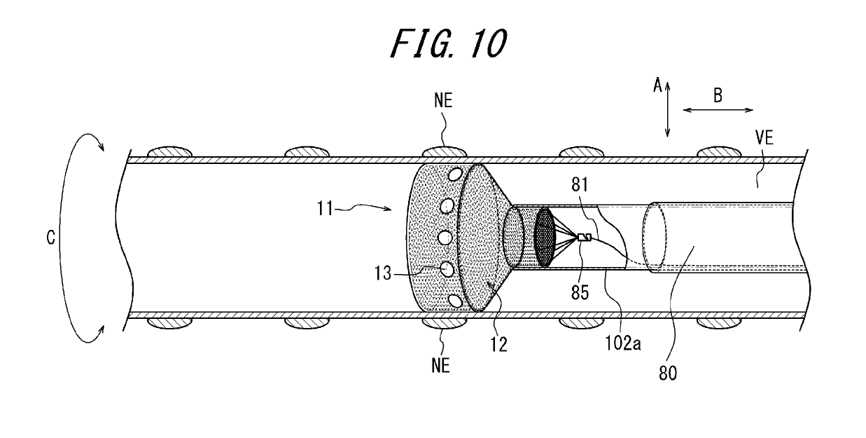

First, the operator inserts the monitoring device 1 into the guiding catheter 80, and pushes the monitoring device 1 into the renal artery RA. That is, the monitoring device 1 is delivered to a predetermined position inside the renal artery RA beyond the distal end 82 of the guiding catheter 80, and causes the expansion body 2 to expand at the predetermined position. The expanded expansion body 2 brings the electrode 3 attached to the surface of the expansion body 2 into a state where the electrode 3 is in contact with the inner wall of the renal artery RA. In addition, the monitoring device 1 is caused to indwell the renal artery RA by using, for example, frictional force generated by the contact between the electrode 3 and/or the expansion body 2 and the inner wall of the renal artery RA. In this state, the electrode 3 can detect the neural activities of the sympathetic nerve located outside the renal artery RA, and can electrically monitor the neural activities. Means for delivering the monitoring device to the predetermined position will be described later (refer to FIG. 10).

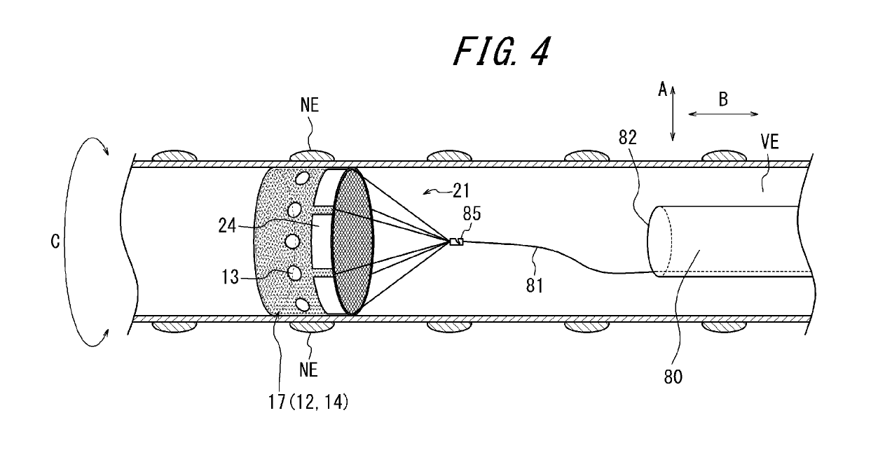

Subsequently, when the denervation is performed on an efferent nerve in the renal artery which extends from the central nerve toward the kidney, the operator inserts a cauterizing device 83 serving as the treatment device into the guiding catheter 80, and inserts a distal end 84 thereof into the vicinity of the previously indwelled monitoring device 1 beyond the distal end 82 of the guiding catheter 80. On the other hand, when the denervation is performed on an afferent nerve in the renal artery which extends from the kidney toward the central nerve, the operator inserts the cauterizing device 83 serving as the treatment device into the guiding catheter 80, and inserts the distal end 84 into the peripheral side of the nerve in the renal artery beyond the distal end 82 of the guiding catheter 80 and the previously indwelled monitoring device 1. In these states, the cauterizing device 83 can perform the denervation by emitting cauterizing energy to the sympathetic nerve to be cauterized. In this case, the electrode 3 itself of the previously indwelled monitoring device 1 has contrasting capability, or as will be described later, at least one identification member 24 (refer to FIG. 4) is arranged on the surface of the monitoring device 1. Accordingly, a position of the monitoring device 1 can be recognized, and the cauterizing device 83 can be inserted. Therefore, without any interference between the cauterizing device 83 and the monitoring device 1, manual operation can be safely performed.

After the monitoring device 1 and the cauterizing device 83 are arranged in the above-described states, the operator causes the cauterizing device 83 to emit ultrasound for cauterizing the sympathetic nerve in the renal artery. The monitoring device 1 indwells the vicinity of the treatment target region using the cauterizing device 83. Accordingly, the operator or his or her assistant can monitor the neural activities of the nerve itself which is cauterized by the cauterizing device 83, through the measuring device located outside the body. Therefore, while the operator performs the treatment for denervation, or immediately after the operator completes the treatment, the operator compares measurement data detected by the electrode 3 before and after cauterizing is performed. In this manner, it is possible to easily determine whether or not the denervation performed by means of cauterizing is actually completed. Specifically, when any influence is observed in the neural activities of the sympathetic nerve in the renal artery before and after the cauterizing is performed, it is possible to confirm that the denervation is completed. When proper cauterizing is not performed, no influence is observed, and so it is possible to easily determine a need for additional cauterizing measures. That is, the electrode 3 enables the operator to accurately determine that the denervation is completed during the treatment or immediately after the treatment. Normally, the cauterizing device 83 for performing the cauterizing performs the cauterizing while the cauterizing device 83 is rotated and pulled out. Accordingly, when additional cauterizing is performed, cauterizing for a necessary site may be performed by inserting the cauterizing device 83 into the vicinity of the monitoring device 1 again.

Furthermore, the monitoring device 1 is configured to indwell the vicinity of the lesion site during the treatment. Accordingly, the major portion inside the guiding catheter 80 can be used for the cauterizing device 83, thereby facilitating the operation of the cauterizing device 83.

Here, a “predetermined position” in the method of use will be described. A relatively large number of nerves in the renal artery extend around the renal artery RA along the extending direction B of the renal artery RA. Accordingly, as long as the electrode 3 comes into contact with the inner wall of the renal artery RA, the electrode 3 is located in the vicinity of the sympathetic nerve in the renal artery which is a measurement target located outside the vessel, and can detect the neural activities. However, as described above, according to the method of use, when the denervation is performed on the efferent nerve in the renal artery which extends from the central nerve toward the kidney, a region where the cauterizing device 83 cauterizes the nerve in the renal artery is necessarily secured inside the renal artery RA. Therefore, it is necessary that a position for expanding the expansion body 2, that is, the “predetermined position” is present inside the renal artery RA, and is a position closer to the kidney from the region for performing the cauterizing. In addition, when the denervation is performed on the afferent nerve in the renal artery which extends from the kidney toward the central nerve, it is necessary that the “predetermined position” is present inside the renal artery RA, and is a position closer to an aortic artery from the region for performing the cauterizing.

As described above, the “predetermined position” described in this description is present inside the vessel VE (corresponding to the renal artery RA according to the method of use), and is at least necessarily a position where the electrode 3 can detect the neural activities of the measurement target nerve NE (corresponding to the sympathetic nerve in the renal artery according to the method of use) located outside the vessel VE. However, the specific position is appropriately determined depending on a type of treatment, a treatment device to be used, or the like.

Furthermore, when the sympathetic nerve in the renal artery is cauterized by the cauterizing device 83 according to the method of use, multiple nerves which are present at different positions in a circumferential direction C of the renal artery RA may be cauterized. In this case, the multiple electrodes 3 are preferably configured to be disposed at different positions in the circumferential direction (the same direction as the circumferential direction C) of the expansion body 2 as in the monitoring device 1, for example, according to the present embodiment 1, since it becomes possible with such a configuration to accurately determine whether the denervation is completed at each position in the circumferential direction C of the renal artery RA.

Next, a monitoring device 11 according to Embodiment 2 will be described. FIG. 3 is a view illustrating a state where the monitoring device 11 indwells the vessel VE. The same reference numerals used in Embodiment 1 are given to respective members which are common to Embodiment 1.

The monitoring device 11 according to the present embodiment includes a self-expandable stent 12 serving as the expansion body 2 which expands at the predetermined position inside the vessel VE, and an electrode 13 which is attached onto an outer peripheral surface of the stent 12, and which detects the neural activities of the nerve located outside the vessel VE by coming into contact with the inner wall of the vessel VE when the stent 12 expands.

The self-expandable stent 12 according to the present embodiment is a covered stent which includes a stent body 14 and a cylindrical cover 15 which covers the periphery of the stent body 14.

The stent body 14 is configured to include a frame structure body 16, and includes a main section 17 of the stent body 14 which has a substantially cylindrical shape as a whole, and a substantially conical connection portion 18 which is continuously disposed in one end portion of the main section 17 having the substantially cylindrical shape. The main section 17 has a substantially cylindrical shape, and a substantially columnar hollow portion 19 partitioned thereinside. Accordingly, even during treatment, a body fluid can pass through the hollow portion 19. In addition, multiple openings 20 are partitioned on a substantially cylindrical outer peripheral surface of the main section 17 by a pattern (design) formed by the frame structure body 16. In the present embodiment, the pattern on the outer peripheral surface of the main section 17 which is formed by the frame structure body 16 has a lattice shape, but may be formed in a spiral shape, a knitted woven shape, or other shapes. The main section 17 of the stent body 14 according to the present embodiment has a substantially circular section. Accordingly, the configuration is advantageously adopted in that the main section 17 is easily inserted (collected) into the guiding catheter which has a substantially circular section (refer to FIG. 10).

In a state where one end portion of the main section 17, specifically, the stent 12 indwells the vessel VE, the connection portion 18 is continuously disposed in one end portion on a side close to the distal end 82 of the guiding catheter 80 within the main section 17. The connection portion 18 is used when the stent 12 is collected after treatment or when the wire 81 (not illustrated) of the electrode 13 is pulled and drawn, and does not press the inner wall of the vessel VE even when the stent 12 expands. Although not illustrated in FIG. 3, the wire 81 is pulled and drawn along the frame structure body 16 configuring the connection portion 18 or by being wound around the frame 16. A hook member 85 is attached to a peak of the connection portion 18, but the hook member 85 is used in collecting the stent 12 after treatment. The collecting method will be described later.

In addition, the connection portion 18 according to the present embodiment is configured so that the frame structure body 16 configuring the main section 17 is extended from one end portion of the main section 17. However, the connection portion 18 may be configured so that another frame member different from the frame structure body 16 configuring the main section 17 is attached to one end portion of the main section 17. In this case, for example, the connection portion 18 can be configured to include a very flexible string member which is formed of various fibrous materials. Furthermore, a configuration can be adopted in which the string member and another member (for example, a metal member) are combined with each other. A material for configuring the connection portion 18 can be the same material as a material for configuring the main section 17, or can be a different material from the material for configuring the main section 17. Specifically, as the material for configuring the connection portion 18, it is possible to use the same material as the material used as the frame structure body 16 (to be described later) or other fibrous materials.

The material of the frame structure body 16 includes a synthetic resin, metal, or the like. As the synthetic resin, for example, polyolefin, polyester, a fluorine resin, or the like can be used. These materials may be used alone, or may be used in combination of two or more materials. Without being particularly limited, polyolefin can be appropriately selected depending on a purpose of use, and includes polyethylene, polypropylene, and the like. In addition, without being particularly limited, polyester can be appropriately selected depending on a purpose of use, and includes polyethylene terephthalate, polybutylene terephthalate, and the like. Similarly, without being particularly limited, the fluorine resin can be appropriately selected depending on a purpose of use, and includes polytetrafluoroethylene (PTFE), a copolymer of tetrafluoroethylene and ethylene (ETFE), and the like. As other characteristics, it is preferable that the fluorine resin is a resin having predetermined hardness and elasticity or a resin having biocompatibility.

In addition, as the metal, it is possible to use stainless steel, a tantalum-titanium alloy, a nickel-titanium alloy, elastic metal, and the like, for example. These materials may be used alone, or may be used in combination of two or more materials. Among these materials, it is preferable to use the elastic metal, and furthermore it is more preferable to use a super-elastic alloy. The super-elastic alloy is generally a so-called shape memory alloy, and exhibits elasticity at a biological temperature (approximately 37° C.) at least. Although not particularly limited, as the super-elastic alloy, it is preferable to use a titanium-nickel alloy containing nickel of 49 atomic % to 53 atomic %.

Without being particularly limited, buckling strength (yield stress when a load is applied) of the super-elastic alloy can be appropriately selected depending on a purpose of use, and is preferably 3 kg/mm² to 20 kg/mm² (22° C.).

Without being particularly limited, recovery stress (yield stress when a load is not applied) of the super-elastic alloy can be appropriately selected depending on a purpose of use, and is preferably 3 kg/mm² to 180 kg/mm² (22° C.).

The “super-elasticity” means that a normal metal recovers a substantially original shape without being necessarily heated after the metal is released from deformation, even when the normal metal is deformed (bent, tensed, or compressed) at an operating temperature so as to reach a plastically deformed region.

In general, in order to prevent body tissues from invading the inside of the stent body 14 from the opening 20 of the main section 17 of the stent body 14, the cylindrical cover 15 is attached to the outer peripheral surface of the stent body 14 so as to cover the periphery of the main section 17. According to the present embodiment, the cylindrical cover 15 is attached to only the outer peripheral surface of the main section 17. However, the cylindrical cover 15 may be attached to at least the outer peripheral surface, or may be configured to be attached to both the outer peripheral surface and the inner peripheral surface. In addition, according to the present embodiment, the cylindrical cover 15 is disposed in an entire region on the outer peripheral surface of the main section 17. However, the cylindrical cover 15 may be configured to be disposed in a portion of the outer peripheral surface.

The thickness of the cylindrical cover 15 is 4 μm to 50 μm, and is particularly preferably 6 μm to 20 μm.

As the material of the cylindrical cover 15, it is preferable to use rubber, elastomer, or a flexible resin. As the rubber, for example, it is preferable to use silicone rubber, latex rubber, or the like. As the elastomer, for example, it is preferable to use fluororesin elastomer, polyurethane elastomer, polyester elastomer, polyamide elastomer, polyolefin elastomer (for example, polyethylene elastomer, polypropylene elastomer), or the like. As the flexible resin, it is preferable to use polyurethane, polyester, polyamide, polyvinyl chloride, ethylene-vinyl acetate copolymer, polyolefin (for example, polyethylene, polypropylene, ethylene-propylene copolymer), or the like.

As a method of attaching the cylindrical cover 15 to the main section 17 of the stent body 14, a film which is produced in advance as the cylindrical cover 15 is joined to the outer peripheral surface of the main section 17 by means of adhesion or the like, for example. As an adhesive used when the film is joined to the main section 17, it is preferable to use those which excellently adhere to the main section 17. For example, when a silicone-based material is used as the configuration material of the cylindrical cover 15, it is preferable to use silica-based primer as the adhesive. When the elastomer is used as described above, it is preferable to use an epoxy resin-based adhesive.

The electrode 13 has a substantially circular flat shape, and is attached to the stent 12. Specifically, the electrode 13 is attached to a surface of the cylindrical cover 15 mounted on the outer peripheral surface of the main section 17 of the stent body 14 by means of adhesion, for example. In particular, it is preferable that within the frame structure body 16, the electrode 13 is attached to a portion whose deformation amount is relatively small before and after the stent 12 expands, or is attached to the surface of the cylindrical cover 15 within a portion which is not deformed. According to this configuration, when the stent 12 expands or contracts, poor connection such as detachment or the like is less likely to occur between the stent 12 and the electrode 13.

FIG. 3 does not illustrate the wire 81 which is connected to the electrode 13. However, similarly to Embodiment 1 described above, the wire 81 is connected to each electrode 13. The wire 81 connected to the electrode 13 is arranged along the outer surface of the cylindrical cover 15, and is pulled and drawn along the frame structure body 16 configuring the connection portion 18 of the stent body 14, or by being wound around the frame structure body 16. A configuration may be adopted in which the wire 81 is pulled and drawn so as to pass through the substantially columnar hollow portion 19 partitioned by the main section 17 of the stent body 14.

In a state where the stent 12 is contracted and size-reduced, the stent 12 having the electrode 13 attached thereto is inserted into the guiding catheter 80 from the outside of the body, and is delivered to a predetermined position inside the vessel VE. The stent 12 expands and indwells at the predetermined position by the electrode 13 attached to the outer peripheral surface of the stent 12 coming into contact with the inner wall of the vessel VE. In this manner, the stent 12 is brought into a state capable of detecting neural activities of the nerve NE. As described above, since the stent 12 according to the present embodiment is a self-expandable type, it is necessary to provide a member which delivers the stent 12 to the predetermined position while the size-reduced state is maintained. The delivery member and the delivery method will be described later (refer to FIG. 10).

According to the present embodiment, the cylindrical cover 15 is disposed in the outer periphery of the stent body 14. However, a configuration may be adopted in which instead of the cylindrical cover 15, a film in which a thin film electrode is formed by using a sputtering method is wound around the outer periphery of the stent body 14. For example, when using materials which are made of polyimide and are not flexible as the film, a configuration is adopted in which the film in a corrugated state is wound before the stent body 14 expands, and in which when the stent body 14 expands, the stent body 14 presses the inner surface of the film so as to spread out the film. In addition, when the film and the electrode which are flexible are used, the film having the electrode formed therein may be wound so as to closely adhere to the outer peripheral surface of the stent body 14 before the stent body 14 expands. The electrode which is flexible can be formed by using a conductive polymer, for example.

Next, a monitoring device 21 according to Embodiment 3 will be described. FIG. 4 is a view illustrating a state where the monitoring device 21 indwells the vessel VE. The same reference numerals used in Embodiment 1 or 2 are given to respective members which are common to Embodiment 1 or 2.

The monitoring device 21 according to Embodiment 3 is different from the monitoring device 11 according to Embodiment 2 described above in that there is provided an identification member 24 which can identify each of the multiple electrodes 13. In the monitoring device 21 according to Embodiment 3, the multiple electrodes 13 are arranged in the circumferential direction C of the main section 17 of the stent body 14. Accordingly, it is possible to concurrently detect and monitor neural activities of multiple nerves located at different positions in the circumferential direction C of the vessel VE. Since the identification members 24 which can mutually identify the multiple electrodes 13 are provided, an operator or the like can perform treatment in accordance with the positions in the circumferential direction C of the vessel VE, based on the obtained data relating to the neural activities.

Specifically, for example, when the above-described treatment for denervation is performed on the sympathetic nerve in the renal artery RA, the neural activities of the multiple nerves which are present at different positions in the circumferential direction C of the renal artery RA are concurrently monitored, and the neural activities before and after cauterizing is performed are compared with each other. As a result of the comparison, if the multiple electrodes 13 cannot be mutually identified when an effect is not observed in only the neural activity detected by one electrode 13 before and after the treatment, an operator cannot visually specify a position of one electrode 13 corresponding thereto, that is, a specific position which needs the treatment for cauterizing again. Therefore, since the identification members 24 which can mutually identify the multiple electrodes 13 as in the present embodiment are provided, the operator can perform the cauterizing again as treatment in accordance with the positions in the circumferential direction C of the vessel VE, based on data relating to the neural activities which are detected by the electrodes 13.

The identification member 24 may be disposed so as to identify the electrodes 13 one by one. However, the multiple electrodes 13 may be included in one group, and one identification member 24 may be disposed for each group. In FIG. 4, one identification member 24 is disposed for two electrodes 13. However, the number of electrodes 13 corresponding to one identification member 24 can be appropriately determined depending on types of treatment, required monitoring accuracy, or the like. For example, in a case of the above-described treatment for denervation, if accuracy in the cauterizing using the cauterizing device 83 (refer to FIG. 2) is considered, it is preferable to dispose at least four identification members 24 at each central angle of 90° in the circumferential direction C. Accordingly, one to approximately several electrodes 13 correspond to one identification member 24.

It is preferable that a position for attaching the identification member 24 is on the surface of the stent 12 and is located in the vicinity of the corresponding electrode 13. In addition, as the material of the identification member 24, it is possible to use a metal piece. For example, the respective identification members 24 can be distinguished from each other on an X-ray image by differently setting shading, shapes, letters, sizes, and the like between the multiple identification members 24.

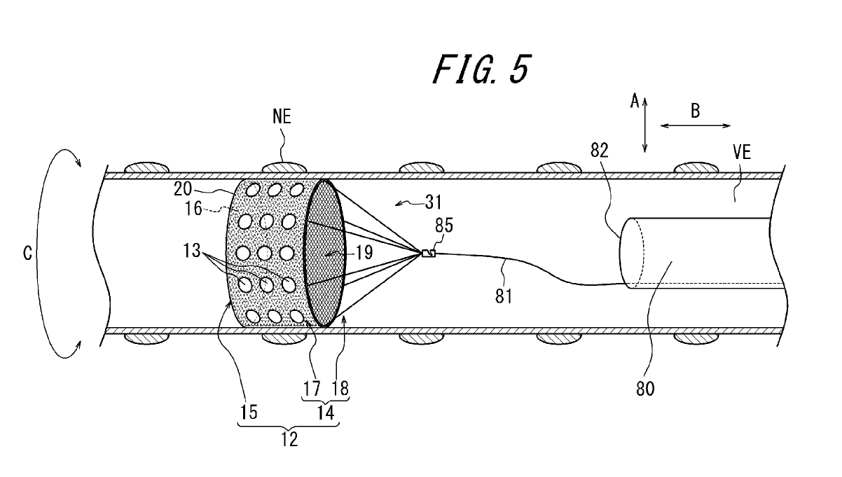

Next, a monitoring device 31 according to Embodiment 4 will be described. FIG. 5 is a view illustrating a state where the monitoring device 31 indwells the vessel VE. The same reference numerals used in Embodiments 1 to 3 are given to respective members which are common to any one of Embodiments 1 to 3.

The monitoring device 31 according to Embodiment 4 is different from the monitoring device 11 according to Embodiment 2 described above in that the electrode 13 is disposed at multiple locations in the extending direction B of the vessel VE. According to this configuration, compared to a configuration in which one electrode 13 having a circular flat shape is disposed in the extending direction B of the vessel VE, the nerve whose neural activities are detectable can be found with a higher probability.

A monitoring device 41 according to Embodiment 5 will be described. FIG. 6 is a view illustrating a state where the monitoring device 41 indwells the vessel VE. The same reference numerals used in Embodiments 1 to 4 are given to respective members which are common to any one of Embodiments 1 to 4.

The monitoring device 41 according to Embodiment 5 is different from the monitoring device 11 according to Embodiment 2 described above in that an electrode 43 has a rectangular flat shape, and in that there is provided a protective filter 44 for protecting the periphery of the vessel VE.

The electrode 43 has a rectangular shape which is elongated in the extending direction B of the vessel VE. Accordingly, compared to the electrode 13 having a circular flat shape, the nerve whose neural activities are detectable can be found with a higher probability. Therefore, the electrode 43 is advantageously employed. In addition, compared to a configuration in which the electrode 13 having the circular flat shape is disposed at multiple locations in the extending direction B of the vessel VE illustrated in Embodiment 4, only one electrode 43 according to the present embodiment may be disposed in the extending direction B of the vessel VE. Accordingly, the number of electrodes to be used decreases. Therefore, it is possible to prevent monitoring from being complicated due to an increased number of electrodes.

The protective filter 44 is attached to an end portion on the downstream side (left side in FIG. 6) in the flowing direction of the body fluid in the vessel VE. For example, if the renal artery RA is assumed as the vessel VE, since the protective filter 44 is attached to the end portion on the downstream side of the stent 12, for example, even when plaque (masses of fat or the like accumulated inside the blood vessel) is detached from the inner wall of the renal artery RA, the protective filter 44 prevents the plaque or the like from entering the blood vessel present in the periphery of the renal artery RA or the kidney. The body fluid such as the blood and the like can pass through the protective filter 44.

For example, as the protective filter 44, it is possible to use a mesh filter having woven metal wires or nylon wires, a membrane filter formed of a polymer having many pores, or the like. It is preferable that the size of the opening in the mesh or the pore is 100 μm or greater.

Next, a monitoring device 51 according to Embodiment 6 will be described. FIG. 7 is a view illustrating a state where the monitoring device 51 indwells the vessel VE. The same reference numerals used in Embodiments 1 to 5 are given to respective members which are common to any one of Embodiments 1 to 5.

The monitoring device 51 includes a balloon 52 serving as the expansion body 2 which expands at a predetermined position inside the vessel VE, and an electrode 53 which is attached to the balloon 52 and detects the neural activities of the nerve located outside the vessel VE by coming into contact with the inner wall of the vessel VE when the balloon 52 dilates.

The balloon 52 has a donut shape, and partitions a hollow portion 54 having a substantially columnar shape. Therefore, even in a state where the balloon 52 dilates and indwells at the predetermined position, the body fluid can pass through the hollow portion 54. In addition, the balloon 52 partitions an annular cavity portion 55, and a liquid is supplied into the annular cavity portion 55, thereby dilating the balloon 52.

The balloon 52 includes a tubular member 56 which communicates with the annular cavity portion 55. The tubular member 56 extends outward from the body through the inside of the guiding catheter 80, and is connected to a syringe located outside the body. Then, a liquid inside the syringe is supplied to the annular cavity portion 55 through the tubular member 56, thereby dilating the balloon 55. On the other hand, the liquid inside the annular cavity portion 55 is withdrawn by the syringe. In this manner, the balloon 55 deflates or is folded and size-reduced. FIG. 7 illustrates the balloon 52 which dilates at a predetermined position by the liquid being supplied into the annular cavity portion 55, and which indwells at the predetermined position.

The balloon 52 can be configured to include an elastically deformable material. However, it is also possible to use a resin-based material which is not elastically deformed, such as nylon, polyethylene, polyether, or polyethylene terephthalate, by forming the resin-based material into a film shape and folding the resin-based material.

According to the present embodiment, the balloon 52 serves to partition the hollow portion 54. However, as described in Embodiment 1, it is also possible to use a balloon which does not partition the hollow portion 54 depending on types of treatment.

In addition, the balloon 52 can adjust a dilatable degree depending on an amount of the liquid to be supplied into the annular cavity portion 55. Accordingly, the balloon 52 is advantageously employed in that the balloon 52 can correspond to an individual difference in diameters of the blood vessel.

The electrode 53 is disposed at multiple locations in the circumferential direction of the balloon 52 in a state where the balloon 52 dilates, and is configured to include a projection portion 57 which is attached onto an outer peripheral surface of the balloon 52 and which is elastically deformable, and a substantially spherical detecting element 58 which is attached to a distal end of the projection portion 57. The projection portion 57 is configured to include metal such as a shape memory alloy and the like. According to the present embodiment, the projection portion 57 configures a portion of the wire 81. The detecting element 58 is an element for detecting the neural activities of the nerve NE which is a measurement target outside the vessel VE by coming into contact with the inner wall of the vessel VE. In FIG. 7, the wires 81 which connect the projection portion 57 of each electrode 53 and a measuring device located outside the body are bundled into one between the monitoring device 51 and the distal end 82 of the guiding catheter 80 in the extending direction B of the vessel VE. However, the wires 81 may be bundled into one in a region where the monitoring device 51 is present in the extending direction B of the vessel VE. The wires 81 bundled into one extend outward from the body through the guiding catheter 80, and are connected to the measuring device.

As illustrated in FIG. 7, it is preferable that the projection portion 57 tilts in a flowing direction D side of the body fluid inside the vessel VE. According to this configuration, compared to a configuration in which an obtuse angle is formed with respect to the flowing direction D of the body fluid inside the vessel VE, the detecting element 58 is configured to sink into the inner wall of the vessel VE. Accordingly, the monitoring device 51 is less likely to be moved by a flow of the body fluid or other external forces.

The projection portion 57 and the detecting element 58 are configured to include metal such as platinum, iridium, tungsten, and the like. The projection portion 57 and the detecting element 58 according to the present embodiment are respectively molded as separate bodies. Thereafter, both of these are connected to each other so as to mold one electrode 53. However, both of these may be integrally molded from the beginning. Furthermore, according to the present embodiment, the projection portion 57 is used as a portion of the wire 81. However, a configuration can also be adopted in which the projection portion 57 is not used as a portion of the electrode 53 by performing an insulating process between the detecting element 58 and the projection portion 57. In a case of this configuration, it is necessary to connect a separate wire 81 to the detecting element 58.

In a state where the balloon 52 deflates and is folded and size-reduced, the balloon 52 having the electrode 53 attached thereto is inserted into the guiding catheter 80 from the outside of the body, and is delivered to a predetermined position inside the vessel VE. The balloon 52 dilates at the predetermined position by the liquid being supplied thereto, and the detecting element 58 of the electrode 53 attached to an outer wall of the balloon 52 comes into contact with the inner wall of the vessel VE. Furthermore, elastic force of the projection portion 57 presses the detecting element 58 against the inner wall of the vessel VE, thereby allowing the monitoring device 51 to indwell. The monitoring device 51 is brought into a state where the detecting element 58 can detect the neural activities of the measurement target nerve NE. According to the present embodiment, as long as the detecting element 58 is in contact with the inner wall of the vessel VE, it is not necessary to bring the projection portion 57 or the outer wall of the balloon 52 into contact with the vessel VE. It is necessary to provide a member which delivers the balloon 52 according to the present embodiment to a predetermined position. However, the delivery member and the delivery method will be described later (refer to FIG. 10).

Next, a monitoring device 61 according to Embodiment 7 will be described. FIG. 8 is a view illustrating a state where the monitoring device 61 indwells the vessel VE. The same reference numerals used in Embodiments 1 to 6 are given to respective members which are common to any one of Embodiments 1 to 6.

The monitoring device 61 according to the present embodiment is configured to include a spiral shape memory alloy 62 serving as the expansion body 2, and an electrode 63 which is attached to a surface of the spiral shape memory alloy 62 and which comes into contact with the inner wall of the vessel VE when the shape memory alloy 62 expands.

Since the shape memory alloy 62 has a spiral shape, the shape memory alloy 62 internally partitions a substantially columnar hollow portion 64. Therefore, the body fluid can pass through the hollow portion 64 of the shape memory alloy 62. In addition, the electrode 63 is arranged at multiple locations with a predetermined distance therebetween in a wiring direction E (direction along a wire configuring the shape memory alloy 62) of the spiral shape memory alloy 62. According to this configuration, the multiple electrodes 63 can be configured to come into contact with the inner wall of the vessel VE at mutually different positions in the circumferential direction C of the vessel VE, and the multiple electrodes 63 can be configured to be disposed at multiple locations in the extending direction B of the vessel VE. A shape of the electrode 63 is a small column shape in the present embodiment. However, without being limited thereto, various shapes such as a circular flat shape, a rectangular flat shape, a spherical shape, and the like can be employed.

As means for attaching the electrode 63 to the shape memory alloy 62, adhering or the like can be performed. Alternatively, a configuration can also be adopted in which a thin film electrode is formed in a film and is wound around the shape memory alloy 62.

In addition, FIG. 8 does not illustrate a relationship between the wire 81 of each electrode 63 and the shape memory alloy 62. However, the wire 81 of the electrode 63 according to the present embodiment is arranged along or by being wound around the shape memory alloy 62. Furthermore, the hook member 85 used when collecting the monitoring device 61 after treatment is disposed in one end portion of the shape memory alloy 62. The hook member 85 will be described later.

The spiral shape memory alloy 62 having the electrode 63 attached thereto is elastically deformed and contracted. In a reduced-size state, the shape memory alloy 62 is inserted into the guiding catheter 80 from the outside of the body, and is delivered to a predetermined position inside the vessel VE. The spiral shape memory alloy 62 expands at the predetermined position due to elastic restoring force. The electrode 63 attached to the surface of the shape memory alloy 62 comes into contact with and presses the inner wall of the vessel VE. The pressing force causes the monitoring device 61 to indwell at the predetermined position, and the monitoring device 61 is in a state where the electrode 63 can detect the neural activities of the measurement target nerve NE. It is necessary to provide a member which delivers the spiral shape memory alloy 62 according to the present embodiment to the predetermined position. However, the delivery member and the delivery method will be described later (refer to FIG. 10).

As described above, the monitoring device can be realized by adopting various specific configurations, and is not limited to the configuration illustrated in the above-described embodiments. Hitherto, in order to facilitate description, some characteristic portions in each of Embodiments 2 to 7 have been described. However, as a matter of course, another configuration can be adopted by combining the configurations described in Embodiments 2 to 7 with each other. For example, as a matter of course, a configuration can be adopted in which the identification member 24 (refer to FIG. 4) according to Embodiment 3 is attached to the monitoring devices except for the monitoring device according to Embodiment 3, or a configuration can also be adopted in which an arrangement and a shape (refer to FIGS. 5 and 6) of the electrode which are illustrated in Embodiment 4 or Embodiment 5 are applied to the electrode according to the other embodiments. Furthermore, as a matter of course, for example, a configuration can be adopted in which the protective filter 44 (refer to FIG. 6) illustrated in Embodiment 5 is attached to the monitoring devices illustrated in the other embodiments, or a configuration can also be adopted in which the electrode (refer to FIG. 7) having the projection portion 57 illustrated in Embodiment 6 is applied to the electrodes according to the other embodiments. In this way, configuring a new monitoring device by combining configurations described in each embodiment with each other is included in the technical scope of the present disclosure.

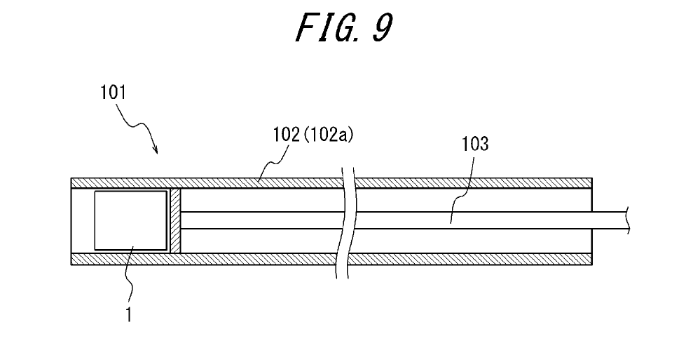

Next, a monitoring device kit 101 including the monitoring device 1 according to Embodiment 1 described above will be described. FIG. 9 illustrates the monitoring device kit 101.

The monitoring device kit 101 includes the monitoring device 1 and a delivery member 102 which delivers the expansion body 2 to a predetermined position by accommodating the expansion body 2 in a state where the maximum length of the expansion body 2 in the radial direction A of the vessel VE is shorter than the maximum length in a state where the expansion body 2 expands at the predetermined position.

The delivery member 102 in a state of internally accommodating the monitoring device 1 is inserted into the guiding catheter 80 from the outside of the body, and delivers the monitoring device 1 into the vessel VE beyond the distal end 82 (refer to FIG. 2 or the like) of the guiding catheter 80. Then, the monitoring device 1 is released from the delivery member 102 at the predetermined position inside the vessel VE, and the expansion body 2 of the monitoring device 1 is expanded so as to indwell at the predetermined position.

In this state, treatment such as cauterizing or the like for the above-described denervation is performed. The monitoring device 1 is contracted or folded and size-reduced after the treatment so as to be accommodated again in the delivery member 102. Thereafter, the monitoring device 1 is drawn outward from the body through the guiding catheter 80.

For example, the delivery member 102 includes a substantially cylindrical outer cylinder member 102a which internally accommodates the expansion body 2. More specifically, it is possible to use a catheter for delivery. It is preferable to configure an outer diameter of the outer cylinder member 102a so as to be smaller than an inner diameter of the guiding catheter 80, and it is preferable that the outer cylinder member 102a is formed of a resin material which is hard to some degree. According to this configuration, it is possible to insert the outer cylinder member 102a into the guiding catheter 80, and it becomes easy to push the outer cylinder member 102a forward to the predetermined position inside the vessel VE. Furthermore, the monitoring device 1 accommodated inside the outer cylinder member 102a is extruded from the outer cylinder member 102a at the predetermined position inside the vessel VE by using an extruding member 103, for example. In this manner, it is possible to easily release the monitoring device 1.

The outer cylinder member 102a has a simple configuration, and is preferably used as the delivery member 102. However, the delivery member 102 is not limited to the outer cylinder member 102a as long as any member can deliver the monitoring device 1 to the predetermined position inside the vessel VE.

Next, with regard to the monitoring devices in Embodiments 2 to 7 in which the stent 12, the balloon 52, or the spiral shape memory alloy 62 is used as the expansion body 2 according to Embodiment 1, the delivery method of the specific monitoring device depending on each type of the expansion bodies 2 and the collecting method after treatment or the like will be described.

First, referring to FIG. 10, the delivery method of delivering the monitoring device (corresponding to the monitoring devices according to Embodiments 2 to 5) using the stent 12 as the expansion body 2 to a predetermined position inside the vessel VE, and the collecting method will be described. Here, description will be made using the monitoring device 11 according to Embodiment 2. However, the description is similarly applied to the monitoring devices 21, 31, and 41 according to Embodiments 3 to 5. Furthermore, without being limited to the configurations according to Embodiments 3 to 5, the delivery method and the collecting method (to be described later) can be used as long as a configuration is adopted in which the stent is used as the expansion body 2. The delivery member 102 will be described by exemplifying the above-described outer cylinder member 102a. However, as described above, the delivery member 102 is not limited to the outer cylinder member 102a.

In a contracted and size-reduced state, the stent 12 of the monitoring device 11 is accommodated inside the outer cylinder member 102a. An operator delivers the outer cylinder member 102a to a predetermined position inside the vessel VE through the guiding catheter 80 from the outside of the body. The “contracted and sreduced-size state” of the stent 12 means a state where the maximum length of the stent 12 in the radial direction A of the vessel VE is shorter than the maximum length in a state where the stent 12 expands at the predetermined position. More specifically, the “contracted and reduced-size state” means a state where the maximum outer diameter in the state is smaller than the maximum outer diameter of the stent 12 in a state where the stent 12 expands inside the vessel VE.

After the outer cylinder member 102a accommodating the monitoring device 11 is delivered to the predetermined position, the monitoring device 11 is released from the outer cylinder member 102a. Specifically, as illustrated in FIG. 9, the monitoring device 11 is extruded from the outer cylinder member 102a. In this manner, the stent 12 is subjected to self-expansion due to elastic restoring force generated by the frame structure body 16 formed of the shape memory alloy, for example. The electrode 13 attached to the outer wall of the stent 12 subjected to self-expansion comes into contact with the inner wall of the vessel VE, and frictional force or the like between the inner wall of the vessel VE and the electrode 13 causes the monitoring device 11 to indwell the vessel VE. The outer cylinder member 102a is pulled out from the guiding catheter 80, and a treatment device is supplied into the vessel VE through the guiding catheter 80.

After treatment is performed in this state, the treatment device is pulled out through the guiding catheter 80. Thereafter, the outer cylinder member 102a is inserted again into the vessel VE through the guiding catheter 80.

The hook member 85 is attached to the stent 12, and a wire (not illustrated) inserted from the outside of the body through the inside of the guiding catheter 80 and the inside of the outer cylinder member 102a is hooked by the hook member 85. Next, the wire hooked by the hook member 85 or the wire 81 is gripped so as to fix a position of the monitoring device 11. In that state, the outer cylinder member 102a is further pushed forward. In this manner, the position of the monitoring device 11 is hardly moved, and the stent 12 is accommodated inside the outer cylinder member 102a again while being contracted. The stent 12 is in contact with the inner wall of the vessel VE. Accordingly, if the stent 12 is moved, there is a possibility of damage to the vessel VE. Therefore, it is preferable to use the collecting method of pushing the outer cylinder member 102a forward as described above.

Thereafter, the outer cylinder member 102a in which the stent 12 is accommodated again is pulled out through the guiding catheter 80, thereby completing the collection of the monitoring device 11.

The delivery method of delivering the monitoring device 51 (refer to FIG. 7) according to Embodiment 6 in which the balloon 52 is used as the expansion body 2 to a predetermined position inside the vessel VE, and the collecting method will be described. As long as a configuration is adopted in which a balloon is used as the expansion body 2, the following delivery method and collecting method can be used without being limited to the configuration according to Embodiment 6. In addition, the method of delivering the balloon 52 to the predetermined position inside the vessel VE is the same as the delivery method of the stent 12 illustrated in FIG. 10. Accordingly, description thereof will be omitted herein, and the collecting method after treatment will be described below.

As described in Embodiment 6, the balloon 52 of the monitoring device 51 is connected to the tubular member 56. A dilating liquid is supplied from a syringe located outside the body to the balloon 52 through the tubular member 56. Here, the tubular member 56 is used in order to collect the monitoring device 51.

After treatment is completed, the liquid inside the balloon 52 is easily pulled out by the syringe connected to the tubular member 56. The balloon 52 from which the liquid is pulled out is brought into a deflated state or into a folded state. Accordingly, an operator draws the tubular member 56 from the outside of the body, thereby easily guiding the monitoring device 51 from the distal end 82 of the guiding catheter 80 into the guiding catheter 80. Thereafter, the tubular member 56 is continuously drawn, and the monitoring device 51 is pulled outward from the body, thereby completing the collecting work. The “deflated state or folded state” of the balloon 52 means a state where the maximum length of the balloon 52 in the radial direction A of the vessel VE is shorter than the maximum length in a state where the balloon dilates at a predetermined position.

Here, a reason why the tubular member 56 is not used as the delivery member 102 will be briefly described. The tubular member 56 is a member placed inside the guiding catheter 80 even during treatment. Accordingly, if operability of a treatment device is considered, it is necessary to configure the tubular member 56 so as to include a relatively thin member. If the tubular member 56 is configured to be thin, rigidity thereof decreases. Consequently, in some cases, it becomes difficult to push the monitoring device 51 into the vessel VE. Therefore, according to the above-described delivery method, the outer cylinder member 102a is used as the delivery member 102. However, when the rigidity of the tubular member 56 is relatively high, or when the rigidity of the wire 81 is relatively high, it is also possible to use the tubular member 56 or the wire 81 as the delivery member 102.

The delivery method of delivering the monitoring device 61 (refer to FIG. 8) in which the spiral shape memory alloy 62 is used as the expansion body 2 to a predetermined position inside the vessel VE, and the collecting method are the same as the delivery method of delivering the monitoring device (refer to FIG. 10) in which the stent is used as the expansion body 2, and the collecting method. Since the methods have already been described, detailed description thereof will be omitted herein.

As long as a configuration is adopted in which the spiral shape memory alloy is used as the expansion body 2, it is possible to use the above-described delivery method and collecting method, without being limited to the configuration according to Embodiment 7. In addition, the delivery member 102 is not limited to the outer cylinder member 102a.

The detailed description above describes a monitoring device and a monitoring device kit. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

### REFERENCE SIGNS LIST

1, 11, 21, 31, 41, 51, 61: MONITORING DEVICE
2: EXPANSION BODY
3, 13, 43, 53, 63: ELECTRODE
4: HOLLOW PORTION OF EXPANSION BODY
12: STENT
14: STENT BODY
15: CYLINDRICAL COVER
16: FRAME STRUCTURE BODY
17: MAIN SECTION OF STENT BODY
18: CONNECTION PORTION OF STENT BODY
19: HOLLOW PORTION OF MAIN SECTION OF STENT BODY
20: OPENING
24: IDENTIFICATION MEMBER
44: PROTECTIVE FILTER
52: BALLOON
54: HOLLOW PORTION OF BALLOON
55: ANNULAR CAVITY PORTION
56: TUBULAR MEMBER
57: PROJECTION PORTION
58: DETECTING ELEMENT
62: SPIRAL SHAPE MEMORY ALLOY
64: HOLLOW PORTION OF SPIRAL SHAPE MEMORY ALLOY
80: GUIDING CATHETER
81: WIRE
82: DISTAL END OF GUIDING CATHETER
83: CAUTERIZING DEVICE
84: DISTAL END OF CAUTERIZING DEVICE
85: HOOK MEMBER
101: MONITORING DEVICE KIT
102: DELIVERY MEMBER
102a: OUTER CYLINDER MEMBER
103: EXTRUDING MEMBER
VE: VESSEL
RA: RENAL ARTERY
NE: NERVE
FA: FEMORAL ARTERY
A: RADIAL DIRECTION OF VESSEL VE
B: EXTENDING DIRECTION OF VESSEL VE
C: CIRCUMFERENTIAL DIRECTION OF VESSEL VE
D: FLOWING DIRECTION OF BODY FLUID INSIDE VESSEL VE
E: WIRING DIRECTION OF SPIRAL SHAPE MEMORY ALLOY

What is claimed is:

1. A monitoring device comprising:
an expansion body that is configured to be delivered into a vessel and to expand at a predetermined position; and
an electrode that is attached to the expansion body, and that is configured to detect neural activities of a nerve located outside the vessel by coming into contact with an inner wall of the vessel when the expansion body expands.

2. The monitoring device according to claim 1,
wherein the electrode is disposed at multiple locations, and the multiple electrodes come into contact with the inner wall of the vessel at mutually different positions in a circumferential direction of the vessel.

3. The monitoring device according to claim 2, further comprising:
an identification member that can identify each of the multiple electrodes.

4. The monitoring device according to claim 1,
wherein the expansion body partitions a hollow portion through which a body fluid can pass.

5. The monitoring device according to claim 1,
wherein the expansion body is a stent, and a hook member is disposed in one end portion of the stent.

6. The monitoring device according to claim 5,
wherein the stent includes a stent body which is configured to have a frame structure body, and
wherein the stent body includes a connection portion to which the hook member is attached.

7. The monitoring device according to claim 2,
wherein the expansion body is a stent, and a hook member is disposed in one end portion of the stent.

8. The monitoring device according to claim 7,
wherein the stent includes a stent body which is configured to have a frame structure body, and
wherein the stent body includes a connection portion to which the hook member is attached.

9. The monitoring device according to claim 3,
wherein the expansion body is a stent, and a hook member is disposed in one end portion of the stent.

10. The monitoring device according to claim 9,
wherein the stent includes a stent body which is configured to have a frame structure body, and
wherein the stent body includes a connection portion to which the hook member is attached.

11. The monitoring device according to claim 4,
wherein the expansion body is a stent, and a hook member is disposed in one end portion of the stent.

12. The monitoring device according to claim 11,
wherein the stent includes a stent body which is configured to have a frame structure body, and
wherein the stent body includes a connection portion to which the hook member is attached.

13. The monitoring device according to claim 1,
wherein the electrode includes an elastically deformable projection portion which is attached to the expansion body and a detecting element which is attached to a distal end of the projection portion, and
wherein in a state where the expansion body expands, the projection portion is elastically deformed, and the detecting element is pressed against the inner wall of the vessel.

14. The monitoring device according to claim 2,
wherein the electrode includes an elastically deformable projection portion which is attached to the expansion body and a detecting element which is attached to a distal end of the projection portion, and
wherein in a state where the expansion body expands, the projection portion is elastically deformed, and the detecting element is pressed against the inner wall of the vessel.

15. The monitoring device according to claim 3,
wherein the electrode includes an elastically deformable projection portion which is attached to the expansion body and a detecting element which is attached to a distal end of the projection portion, and
wherein in a state where the expansion body expands, the projection portion is elastically deformed, and the detecting element is pressed against the inner wall of the vessel.

16. The monitoring device according to claim 4,
wherein the electrode includes an elastically deformable projection portion which is attached to the expansion body and a detecting element which is attached to a distal end of the projection portion, and
wherein in a state where the expansion body expands, the projection portion is elastically deformed, and the detecting element is pressed against the inner wall of the vessel.

17. The monitoring device according to claim 5,
wherein the electrode includes an elastically deformable projection portion which is attached to the expansion body and a detecting element which is attached to a distal end of the projection portion, and
wherein in a state where the expansion body expands, the projection portion is elastically deformed, and the detecting element is pressed against the inner wall of the vessel.

18. The monitoring device according to claim 6,
wherein the electrode includes an elastically deformable projection portion which is attached to the expansion body and a detecting element which is attached to a distal end of the projection portion, and
wherein in a state where the expansion body expands, the projection portion is elastically deformed, and the detecting element is pressed against the inner wall of the vessel.

19. A monitoring device kit comprising:
the monitoring device according to claim 1; and
a delivery member that accommodates the expansion body in a state where the maximum length of the expansion body in a radial direction of the vessel is shorter than the maximum length in a state where the expansion body expands at the predetermined position, and that can deliver the expansion body to the predetermined position.

20. A method of monitoring a nerve located outside a vessel, comprising:
delivering an expansion body to a predetermined position within the vessel, said expansion body having an electrode attached thereto;
expanding the expansion body at the predetermined positioned within the vessel, thereby causing the electrode to come into contact with an inner wall of the vessel; and
detecting neural activities of the nerve using the electrode.
